(19) United States
(12) Patent Application Publication
(10) Pub. No.: US 2006/0121512 A1
(43) Pub. Date: Jun. 8, 2006

(54) Biological pathways in progenitor cells

(75) Inventor: Nancy L. Parenteau, Benson, VT (US)

Correspondence Address: OLIFF & BERRIDGE, PLC, P.O. BOX 19928, ALEXANDRIA, VA, 22320, US

(73) Assignee: ORGAN RECOVERY SYSTEMS, INC., Des Plaines, IL (US)

(21) Appl. No.: 11/292,147

(22) Filed: Dec. 2, 2005

Related U.S. Application Data
(60) Provisional application No. 60/632,386, filed on Dec. 2, 2004.

(51) Int. Cl. C12Q 1/68; G01N 33/567; C12N 5/08; G01N 33/53

(52) U.S. Cl. 435/6; 435/7.2; 435/370

(57) ABSTRACT

The disclosure relates to methods of identifying biological processes associated with non-fetal progenitor cells, and of identifying molecules and factors that regulate or modulate these processes, and is useful in fields including regenerative medicine (tissue regeneration), transplantation, and cancer research.

### BACKGROUND

Adult and embryonic stem cells are the subject of intense scientific interest because of their potential role in cell therapies. Cultured populations of such cells, when implanted and properly activated in vivo, can be useful to augment, repair, restore, or replace a diseased, damaged, missing, or otherwise compromised tissue or organ. While several mature cell types in a mammalian body, e.g., liver cells, have the ability to multiply to a greater or lesser extent to meet changing physiological demands or to recover from injury, it is the proliferative tissue stem cell and/or progenitor cell, that renews the cell population as needed. The biology of progenitor cells is important for proper organogenesis in the fetus; moreover, it has the potential to impact organ homeostasis, renewal, abnormal growth, regeneration and aging in the adult. In theory, the more compromised an adult organ is, the more likely its progenitor cell/compartment will be stimulated to proliferate. Study of organ regeneration in response to disease and injury has provided evidence for this response in the liver, however, the behavior of a distinct, expandable progenitor cell compartment in other adult tissues such as skin and the pancreas has remained unclear.

### SUMMARY

The disclosure relates to methods of studying biological pathways, e.g., signaling cascades, in progenitor cell populations in vitro. The progenitor cells may originate from various non-fetal tissues.

Because cellular interaction among the progenitor cells during organogenesis is likely to be only partially applicable to mechanisms of their interaction, activation or regenerative failure within the mature organ, knowledge on parenchymal progenitor interactions and regeneration has to be gained from cells derived from mature organs. However, it has been hard to capture and utilize normal adult progenitor cells until now. This is because, once beyond a stage of organogenesis in the fetus when progenitor cells are naturally abundant, cells in the progenitor state are present to a very limited degree in the normal mature organ and are in need of specific circumstances to foster their proliferation. Also, to be properly assessed, the progenitor cells must be in an “active” state, which is difficult to achieve in vivo.

Cultivation of non-fetal stem and progenitor cells in vitro, including adult developmentally authentic progenitor (ADAP) cells, has been hampered due to difficulties associated with their selective cultivation. For example, a major issue in the establishment of progenitor cell cultures from an adult pancreas or adult islet tissue is the overgrowth of contaminating, non-parenchymal cell types and the continued presence of differentiation-committed cells.

Therefore, little is known about the biological nature of non-fetal stem and progenitor cells and what kind of signaling, both intra- and inter-cellular, that either activates them to expand the progenitor population, retains them in their respective phases of differentiation, or advances them through the various phases and eventually to tissue regeneration. For example, according to one working model, these regenerative phases start from slow-cycling resident stems cells to progenitor cells, then transit amplifying cells, and eventually to maximally differentiated cells, such as parenchymal cells.

There is a need for the ability to identify, access, and manipulate ADAP cells cultivated from cell cultures. Further, there exists a need for information on the biological pathways that generate and regulate a cell culture with the majority of the cells being ADAP cells capable of prolonged expansion in vitro and organ regeneration with high fidelity in vivo. That information includes cellular activities of these and other cells in between the developmental phases of stem cells and their fully differentiated offspring. Such information can lead to the identification of drug molecules that modulate these pathways.

The disclosure provides methods of identifying a biological processes associated with non-fetal progenitor cells and compositions identified through such methods. In embodiments, the methods include providing at least one substantially pure population of non-fetal progenitor cells originating from a differentiated tissue, and identifying a biological process present in the population of non-fetal progenitor cells but absent or substantially absent in a differentiated cell or a substantially pure population of non progenitor cells. The methods may also include providing a second substantially pure population of non-fetal progenitor cells originating from a differentiated tissue different from that of the first population of progenitor cells, and identifying a biological process present in both populations of non-fetal progenitor cells but absent or substantially absent in the population of non progenitor cells. An example of non progenitor cells is fibroblast cells.

In embodiments, the methods include providing at least one substantially pure population of non-fetal progenitor cells originating from a differentiated tissue, administering a test molecule to the population of progenitor cells, and identifying a biological process affected in the population of non-fetal progenitor cells upon administration of the test molecule. The methods may also include providing a second substantially pure population of non-fetal progenitor cells originating from a differentiated tissue different from that of the first population of progenitor cells, administering the test molecule to the second population of progenitor cells, and identifying a biological process affected in both populations of non-fetal progenitor cells upon administration of the test molecule. Furthermore, the methods may include identifying a biological process affected in both populations of non-fetal progenitor cells but not affected in a differentiated cell or a substantially pure population of non progenitor cells.

Examples of “biological process” include, but are not limited to, the level of expression of a gene or the amount of a gene product (a messenger RNA or a protein) in the non-fetal progenitor cells upon administration of the test molecule.

In embodiments, the methods include comparing levels of protein and gene expression in progenitor cells to those in more differentiated cells of the same origin. The progenitor cell populations may originate from, for example, parenchymal tissue, such as pancreatic islets, liver hepatocytes, or epidermal cells.

In embodiments, the methods identify at least one test molecule and related biological process common to the propagation or the differentiation of progenitor cells of more than one origin. In other embodiments, the methods identify at least one test molecule and related biological process common to propagation or differentiation of progenitor cells of the same origin.

The methods may include creating a database containing data indicative of the biological process presented or affected in the non-fetal progenitor cells.

The disclosure also provides methods for assaying the activity of a test molecule on non-fetal progenitor cells. The methods may include providing a substantially pure population of non-fetal progenitor cells in a defined cell culture medium, administering a test molecule to the population of non-fetal progenitor cells, and determining if the test molecule modulates a biological process within cells of the cell population. The population of progenitor cells preferably originates from parenchymal tissue, such as from pancreatic islets, liver hepatocytes, or epidermal cells. In embodiments, the test molecule is assayed on a plurality of non-fetal progenitor cell populations for any modulatory effect, each cell population originating from a different and differentiated tissue.

The disclosure also provides a cDNA library that contains a population of DNA molecules representative of genes expressed in a progenitor cell originated from a mature tissue, such as the liver tissue, pancreas tissue, or epidermis

The disclosure also provides a cDNA library that contains a population of DNA molecules representative of genes commonly expressed in at least two populations of progenitor cells where each population has originated from a different mature tissue, such as the liver, the pancreas, or the epidermis.

### BRIEF DESCRIPTION OF THE DRAWINGS

FIG. 1 is a schematic representation showing various hypothetical cellular phases in parenchymal neogenesis.

FIG. 2 is a schematic representation showing various embodiments of the disclosed methods.

### DETAILED DESCRIPTION OF EMBODIMENTS

The disclosure provides methods for studying functional native signaling involved in the activation, growth, expansion, and progression from non-fetal stem and progenitor cells to their offspring. Non-fetal cells include neonatal and adult cells, for example, cells from mature parenchyma. In particular, the disclosure teaches the generation and comparative use of adult developmentally authentic progenitor (ADAP) cells and associated biological processes for the discovery, analysis and screening of compounds that affect organ regeneration and related processes.

The disclosure provides a hypothesis-driven, target-down approach to systems biology. Part of the hypothesis is that adult stem and progenitor cells are defined by their behavior and not necessarily by specific genetic markers. Therefore, the exclusive search for genetic markers, an approach that may work in stem cells of embryonic origins, may not be optimal when using adult stem and progenitor cells. Instead, studies of stem and progenitor cells of non-fetal origins and their regulation should take into account expression patterns beyond the genetic level, such as transcription, translation, post-translational modification and relocation. Accordingly, in embodiments, both functional genomics and proteomics of non-fetal stem cells and its offspring in all phases of tissue regeneration are examined.

A challenge in the characterization of non-fetal stem and progenitor cells is the lack of a pool or compartment of progenitor cells that can be readily identified in vivo. Embodiments circumvent this difficulty by establishing a pool of progenitor cells, specifically, a pool of ADAP cells in vitro. This population of progenitor cells originates from non-fetal tissues, e.g., slow-cycling stem cells from adult parenchyma. Past attempts of generating such a progenitor cell pool were compromised with either overgrowth of contaminating non-parenchymal cell types such as stromal or “nurse” cells, or continued presence of more differentiation-committed cells such as transit amplifying cells. In particular, these contaminating cells represented the majority of the cells in the culture. The contaminating cells and other components in the cell culture such as serum, growth factors, cAMP elevating agents, organ extracts, favored a variety of undesirable cell responses in the primary cell population, such as a compensatory growth, uncontrolled partial differentiation, loss of differentiated character with limited growth, or cells undergoing transdifferentiation or an epithelial to mesenchymal transition in an adaptive, metaplastic response. These undesirable cell responses lead to conflicting results, thereby slowing the elucidation of biological mechanisms relating to organ regeneration in adult tissues.

In contrast, in embodiments, populations of ADAP cells are cultivated in vitro into majority by number from cells, such as stem cells, that originate from mature tissue parenchyma. Specifically, a primary cell sample, when taken from an individual and introduced into a stringent environment limited to nutritional and metabolic support for the ensuing apoptotic and regenerative reaction within the explanted tissue sample, can result in the development of a population of ADAP cells. Accordingly, in embodiments, the cells under investigation are placed in a highly or chemically defined culture environment. “Chemically defined” means that the culture medium essentially contains no or substantially no serum or organ extracts. It was not expected that a growing, healthy, e.g., epithelial, cell population can be derived from adult tissues under these stringent conditions or that the resulting cells exhibit properties of the ADAP.

An advantage of using populations of substantially pure progenitor cells cultivated in vitro is that their environment can be strictly controlled and free of potentially confounding in vivo factors. Further, the stringent cell culture may omit all components that have the potential of promoting undesirable cell responses such as sustained compensatory growth of mature cells, in vitro metaplasia and artifactual gene expression.

The methods result in cultures where ADAP cells constitute a majority, or preferably, a higher percentage, e.g., about 60, about 70, or about 80 percent, of the cell population by number. In embodiments, the cell culture is a substantially pure or homogenous population of ADAP cells. Such populations of ADAP cells may be maintained in that regenerative phase and propagated or expanded through serial passages without further differentiation. In embodiments, such populations of ADAP cells are allowed to progress to the subsequent phases of regeneration, i.e., into transit amplifying cells and fully differentiated cells. Cells from any phase of the regeneration may be added to a suitable carrier to be administered into an individual for clinical treatment or therapy.

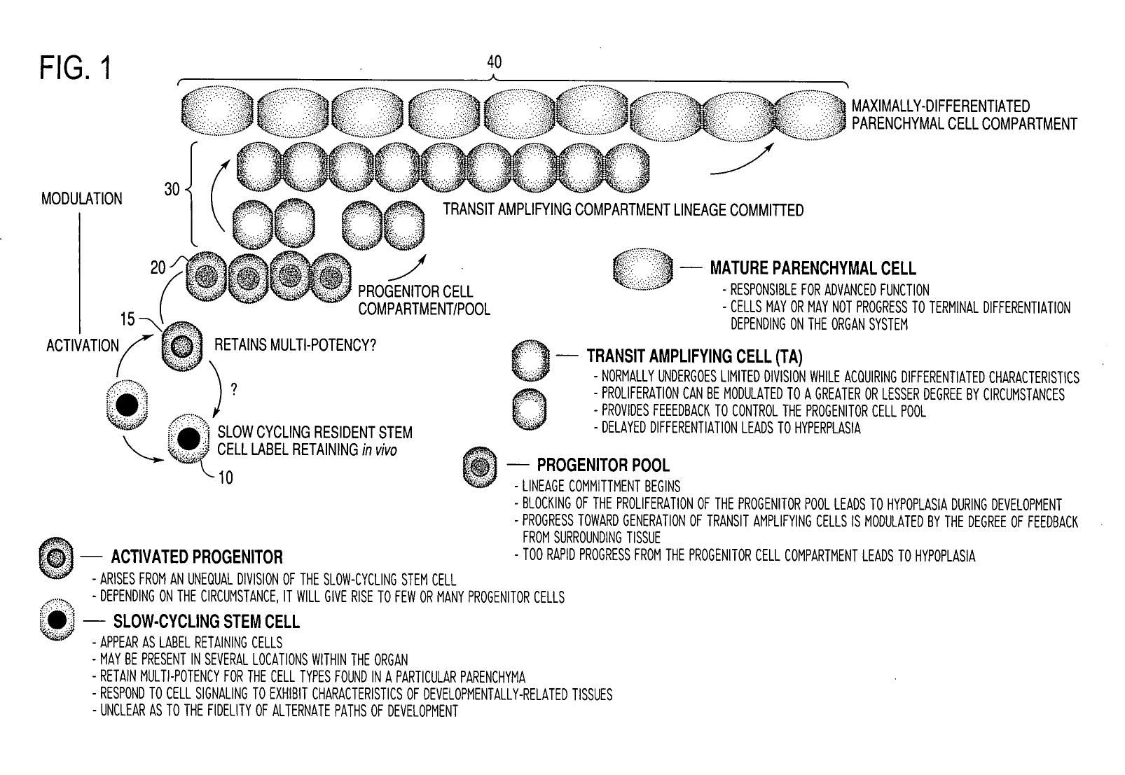

Referring to FIG. 1, to achieve neogenesis, i.e., de novo generation of functional tissue, embodiments focus on identifying biological pathways involved in tissue regeneration. In embodiments, a primary cell culture derived from an organ, e.g., the pancreas, contains multiple cell types. Resident stem cells 10 are slow-cycling cells that are activated from quiescence to give rise to activated progenitor cells 15 through unequal division. Slow-cycling stem cells 10 retain labels in vivo and may be present in several locations within the organ. The stem cells 10 retain multi-potency for the cell types found in a particular parenchyma. The activated progenitor cells 15, depending on the circumstance, may give rise to few or many progenitor cells 20, or, alternatively, may cycle back to stem cells 10.

Progenitor cells 20, i.e., the ADAP cells, are minimally differentiated cells and make up the proliferating cell compartment responsible for organ regeneration. In embodiments, the ADAP cells are organ-derived cells in a proliferative state that are characterized by (i) a preferential proliferative response to the growth-factor-free, or otherwise chemically defined, media conditions, (ii) their lack of differentiated characteristics, and (iii) the expression of genes characteristic of fetal organ progenitor cells seen during development. The ADAP cells 20 can generate transit amplifying (TA) cells 30, which, in turn, lead to parenchymal cells 40. The fact that ADAP cells retain the ability to generate TA cells capable of recapitulating the normal developmental process of organogenesis distinguishes them from cells undergoing a compensatory growth response, more distantly related cells undergoing an adaptive metaplasia, and cells undergoing an epithelial-to-mesenchymal transition, all of which have been implicated as putative or facultative progenitor cells.

While lineage commitment begins in progenitor cells, the TA cells 30 are lineage committed, differentiating cells, and exhibit limited replication. The parenchymal cells 40 are maximally differentiated functional cells. Other researchers have described the same or a similar process with or without the activated progenitor cells 15 and/or progenitor/ADAP cells 20 altogether. The present invention should not be limited to the embodiment depicted in FIG. 1 and should instead be understood for the principle depicted and described herein.

The derivation of ADAP cell populations permits the acquisition of gene and protein information free of confounding factors during the first steps in organ renewal, the activation and proliferation of a tissue progenitor population. Being able to distinguish regenerative signaling involving developmental progenitor regulation from compensatory regrowth and forms of metaplasia is important to the discovery and testing of molecules that directly impact organ renewal.

Referring back to FIG. 1, the process of regeneration is presumably activated as slow-cycling stem cells 10 respond to cell signaling. The remainder of the regeneration process contains events that are presumably regulated through signaling events. For example, progress of progenitor cells 20 towards generating TA cells 30 has been observed to be modulated by the degree of feedback from surrounding tissue. In liver, for example, a progenitor compartment is not observed except in cases of severe damage or illness. Similarly, proliferation of TA cells 30 appears to be modulated to a greater or lesser degree by circumstances. And the TA cells 30 appear to provide feedback to control the size of progenitor cell pool. Therefore, there are at least three levels of population regulation that are particularly important to regenerative technology: (1) activation of the progenitor cell, (2) control of progenitor cell pool size, and (3) regulation of the TA cells toward acquisition of advanced function.

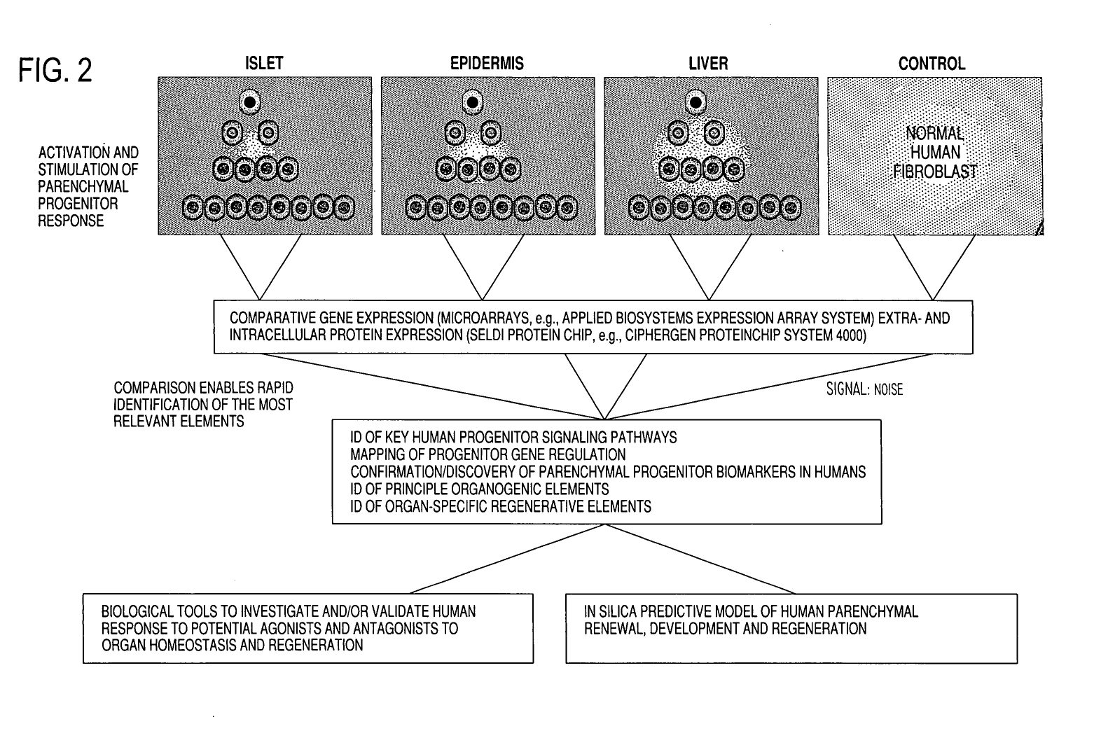

Referring to FIG. 2, to study the functional signaling involved in the regenerative response, ADAP cell populations are first generated in vitro as described above and further detailed in the examples below. In embodiments, ADAP cell populations are generated from different parenchyma: e.g., the Islets of Langerhans, the epidermis, and the liver. Then, these cell populations are analyzed in terms of finctional genomics and proteomics of their native progenitor potentials. In embodiments, the biological definition of the progenitor state is probed by characterizing the expression patterns of proteins, nucleic acids (e.g., RNAs such as mRNA), and other biomolecules in the cell. A control can be normal differentiated tissue cells, for example, fibroblast cells from the same individual, e.g., a human. Comparisons of the above expression patterns among ADAP cells of different tissue origins and against the control cells provide information about biological pathways common to progenitor cells, or alternatively, biological pathways specific to progenitor cells derived from a specific tissue.

For example, a comparison of biomolecules found in active, i.e., expanding, ADAP cells originating from the pancreas and the liver gives information on biomolecules, e.g., expressed proteins, common to progenitor cells in at least those two organs, and maybe to more or even all tissue in the host. Once biomolecules found in the control tissue, e.g., mature fibroblast or differentiated cells of the same organ origin, are subtracted from the biomolecule common to ADAP cells originating from the pancreas and the liver, a better idea of what biomolecules that may define a common progenitor state in the host emerges. With ADAP cells from more tissues/organs added to this comparison, more confidence is given to the resulting list of “common” ADAP-defining biomolecules. A comparison of an ADAP cell's biomolecules with those found in the control provides a list of tissue-specific profile. That profile can be further compared with the “common” list, so that biomolecules unique to the ADAP cells of that tissue can be identified. Information generated from such studies can be entered, compiled, and organized into a searchable database and stored electronically, e.g., on a computer-accessible memory storage space.

While detailed examples are described below, any suitable tools and protocols known to one skilled in the art of genomics and proteomics can be used for purpose of the invention. For example, commercially-available microarrays chips or other high throughput means, such as Applied Biosystem's Expression Array System, can be used to conduct comparative gene expression studies. Commercially-available matrix-assisted laser desorption/ionization (MALDI) or surface enhanced laser desorption/ionization (SELDI) mass spectrometry, such as Ciphergen's ProteinChip System, can be used to conduct protein expression studies. The proteins studied can include extra-cellular, membrane, or intracellular proteins including but not limited to enzymes, lipids and glycoproteins.

In embodiments, signaling pathways affecting these populations of ADAP cells that originate from different tissues are examined and identified. By generating activated progenitor populations from different organs, canonical pathways/relationships of regeneration as well as tissue-specific differences in the pathways can be discovered. Stimulating the regenerative response in vitro rather than attempting to analyze cells and tissues directly from the in vivo environment in a top-down approach, also aids analysis by creating functional progenitor activation, in a highly defined environment free of potentially confounding in vivo factors. Therefore, the “signal” for the study is enhanced against the “noise” by choosing an in vitro and highly defined environment.

In embodiments, normal activation and expansion of the ADAP cells are perturbed with test molecules or factors to study the signaling pathways. Successful activation of stem cells into generation of progenitor cells and their serial expansion have been described in co-owned international application PCT/US2004/17284 filed on Jun. 3, 2004, the entire disclosure of which is incorporated herein by reference. In a preferred embodiment, the cell culture conditions are selected that undermine more differentiated cells, thus releasing the inhibitory influence that more differentiated cells normally have on the growth of progenitor cells. The result is a culture that allows the formation of colonies of self-supporting, undifferentiated progenitor cells that constitute a majority of the cell culture, preferably about 60%, about 70%, or about 80% progenitor cells by number.

Although any set of culture conditions that promote progenitor cell growth are contemplated, an embodiment of a method for propagating progenitor cells includes a serum-free medium that induces apoptosis or necrosis in the differentiating and/or differentiated cells. Cells may be initially cultured in a stringent primary medium with no, or low levels of calcium and/or growth factors, to bias the culture toward progenitor cell activation and growth. After ADAP cell growth has been initiated and the ADAP cells expand to be the majority of the cell population, the cells can be propagated in a secondary, minimal growth medium that can be less stringent than the primary medium. Finally, differentiation of the resulting ADAP cell culture may be promoted by addition of differentiating factors in a tertiary medium in the presence of specific growth factors.

Referring back to FIG. 2, in any of the regenerative phases described above, i.e., between activation of stem cells to the maximum differentiation, one or more test molecules can be added to the defined environment, preferably in a measured amount. Cell responses, including at the behavioral and at the molecular levels, can be compared as before, during, and after the administration of such test molecules in order to map out potential biological pathways regulating or modulating cells involved in regeneration. Such test molecules can include, but are not limited to, growth factors, ligands, cytokines, lymphokines, chemokines, hormones, cAMP, and so on. By studying the expression patterns of proteins, nucleic acids (e.g., RNAs such as mRNA), and other biomolecules in the cells, it is possible to identify pathways, relevant biomarkers expressed or secreted during regeneration, and organogenic elements. All the above information can be entered, compiled, and organized into a searchable database, and stored electronically, e.g., on a computer-accessible memory storage space.

In embodiments, the cultured progenitor population primarily relies on autocrine and paracrine response to bring about activation and expansion of the progenitor. Therefore, signaling and gene expression retains fidelity to native mechanisms. The stem and progenitor cells may derive or originate from any organ or tissue containing parenchymal cells capable of regeneration including, but not limited to, a cell population derived from pancreas, liver, gut, heart, kidney, cornea, skin, retina, inner ear, skeletal muscle, brain, or glands. In embodiments, the population of progenitor cells gives rise to cells of a specific parenchymal lineage, e.g. pancreatic islet endocrine lineage, liver hepatocyte cell lineage, or epidermal cell lineage.

In embodiments, ADAP cell populations can be further used as biological tools to investigate and/or validate the individual's response to potential agonists and antagonists to organ homeostasis and regeneration. Information generated using methods of the invention can be used to produce an in silica predictive model of human or other species' parenchymal renewal, development and regeneration.

The disclosure is further distinguished from a global bioinformatics and systems biology approach in that it enables the manipulation and analytical comparison of the genetics, signaling and behavior of a functional cellular target under activated, defined conditions to produce outputs that are greatly simplified while retaining a high degree of relevance to the adult human condition. Embodiments can be used for discovery of novel genes and proteins, identification of drug targets, novel regulatory pathways, and bioassays to assess impact of agents on regenerative processes.

Cultivating ADAP Cells

In embodiments, a population of substantially pure epithelial progenitor cells is produced in vitro by culturing a primary cell culture of epithelial cells in a primary culture medium that induces a stress response in the cells which depletes mature, differentiated parenchymal cells and/or differentiating transit amplifying cells. This response alters the dynamics of cell signaling in the culture to permit the ADAP cells to replicate and propagate. The stress response kills the more differentiated cells such that the resulting cell population is substantially free of differentiated or differentiating cells, and contaminating cells from other tissue types, e.g., stromal, fibroblast cells. While it is not yet certain, suppression of more differentiated cells may silence cell-to-cell signaling that inhibits the replication of progenitor cells and/or possibly provide signaling to activate progenitor proliferation. As a result, the progenitor cells propagate without any type of “feeder” or “nurse” cells from other tissue types.

There are various other ways to monitor the stress response besides visual observation. For example, the expression of a heat shock protein or an acute phase reactant gene can be measured as an indicator of the stress response.

One way to identify a pre-confluent colony of progenitor cells is to determine whether the primary culture cells are undergoing active mitosis. Other ways include observing the cells under the microscope; or adding 5-bromo-deoxyuridine (BrdU), a thymidine analog, to the cell culture and detecting the incorporation of the BrdU into the cells using a monoclonal anti-BrdU antibody.

After a pre-confluent colony of progenitor cells is identified in the primary culture, it can be separated and used to establish a secondary cell culture comprising substantially homogeneous progenitor cells. The secondary cell culture may use the same type of culture medium as the primary culture, or use a medium that is less stringent. The secondary cell culture maintains the progenitor cells through multiple passages, and the progenitor cells retain the ability to differentiate or undergo neogenesis. In embodiments, the progenitor cells undergo no less than five passages.

In embodiments, the methods may further include steps to activate the progenitor cells to become differentiating cells, e.g., transit amplifying cells, and/or differentiated cells, e.g., parenchymal cells.

Primary Cell Culture

The primary cell culture is designed to induce a stress response in differentiating and differentiated cells including contaminating cells of other tissue types, but to permit progenitor cells to propagate. It is believed that once the differentiating and differentiated cell population becomes depleted, the progenitor cells become activated, enter the cell cycle and start dividing with increasing rapidity. The stress response that initiates this selection process for the progenitor cells may be induced through a variety of means, for example, by inducing the apoptosis and/or necrosis of these cells. In embodiments, the primary culture medium is chemically-defined. “Chemically-defined” means that the culture medium essentially contains no or substantially no serum or organ extracts. In embodiments, the medium contains a low level or is substantially free of growth factors. If a growth factor is present, it is preferably less than about 10 ng/mL, more preferably less than about 5 ng/mL, e.g., about 1 ng/mL. In embodiments, the medium contains cAMP elevating agents, such as cholera toxin and foreskolin, preferably at a concentration of about 9 ng/mL to support the activation and outgrowth of the progenitor cells.

The primary culture medium may be designed to inhibit cell-cell adhesion. For example, the medium may contain nitric oxide, which is known to inhibit cell adhesion and to disrupt cell-matrix interaction. Alternatively or in addition, tumor necrosis factor-alpha (TNF-a), interleukin 1-beta (IL1-13), and interferon-gamma (IFN-y) can be added to stimulate nitric oxide-induced apoptosis. The cells also may be cultured in diluted hydrocolloid, dextran, and the like, to disrupt cell adhesion and to disfavor the survival of more differentiated cells.

In embodiments, the medium contains a low level of or no calcium. If calcium is present in the culture medium, the concentration of calcium is preferably less than about 1 mM, e.g., between about 0.001 to about 0.9 mM. For example, the calcium concentration can be between about 0.01 to about 0.5 mM, or about 0.08 mM. While not wishing to be bound by theory, the low calcium environment is thought to limit the cell-to-cell contact necessary for the interaction and maintenance of the more differentiated cells. A low calcium environment combined with the chemically-defined culture medium and minimal concentration of growth factors causes the differentiated cells to divide more slowly, eventually causing those cells to undergo apoptosis resulting in a population of progenitor cells within the culture.

Many apoptosis or necrosis-related pathways are known in the art. The primary culture medium can be designed to initiate or enhance such pathways. Pathways that down-regulate such stresses can be deactivated—for example, protein kinases that inhibit apoptosis can be blocked. Many of these pathways are cooperative and can be used in combinations. Examples of such signaling pathways include the caspase pathways, the Bcl-2 pathways, and the interleukin-10 pathway.

The caspase pathway involves nuclear factor kappa B (NF-KB) which is a transcription factor that, once translocated to the nucleus, activates transcription of various genes including those affecting the onset of cell death. Ligands, antigens, antibodies, growth factors, cytokines, lymphokines, chemokines, cofactors, hormone and other factors that regulate NF-KB, such as tumor necrosis factor (TNF) can be added to the culture medium to kill the differentiating and/or differentiated cells through the caspase, Bcl-2, and other pathways. Such factors include TNF-a, TNF-like weak inducers of apoptosis (TWEAK), TNF-related apoptosis-inducing ligands (TRAIL), interleukins (IL) (e.g., IL 10), Fas ligands, Apoptosis inducing protein ligands (e.g., APO-3L and 2L), transforming growth factor beta (TGF-(3), endotoxins (e.g., lipopolysaccharide), regulated-upon-activation normal T-cell expressed and secreted (RANTES), interferons (e.g., IFN-y), oxadaic acid (a serine/threonine protein-phosphatase inhibitor) and so on.

Examples of apoptosis-inhibiting signaling pathways that can be disrupted to disfavor the survival of more differentiated cells include the AKT-mediated signaling pathway and those activated by other so-called “survival kinases” such as IKK, erk, Raf-1. Possible ways to interfere with the AKT signaling pathway include use of siRNA, growth factors such as those produced by autocrine/paracrine, and/or antibodies to block the receptor tyrosine kinase AKT. Alternatively, elimination of growth factors that could induce this pathway using a stringent, defined medium may lead to similar results. Another example of disrupting apoptosis-inhibiting signals includes deactivation of heat shock protein 70.

Other environmental factors such as heat, radiation, humidity, and pH also can lead the desired stress response in the cell culture. For example, ultraviolet radiation may induce cell death in more differentiated cells.

Secondary Cell Culture

The secondary cell culture typically comprises progenitor cells selected from the primary cell culture by virtue of their ability to thrive in the stressed conditions. The secondary cell culture maintains the progenitor cells so that they maintain the potential to differentiate or undergo neogenesis without actual differentiation. The ability of a population of progenitor cells to endure prolonged propagation through serial passage brings about another advantage of the present invention, which is to ability to amass a sufficient amount of progenitor cells for neogenesis and other applications.

The secondary cell culture may use the same type of medium as the primary culture as it continues to suppress differentiation of the progenitor cells. Alternatively, the secondary culture medium may be less stringent. Some limited amount of growth factors may be added to the base medium since the culture initially is substantial free of more differentiated cells. Some non-essential growth factors can be used sparingly or intermittently in the secondary culture medium. Examples of such growth factors include epidermal growth factor (EGF), transforming growth factor alpha (TGF-a), keratinocyte growth factor (KGF), and basic fibroblast growth factor.

Further Regulation

Cells harvested from a primary or secondary culture can be further regulated. In embodiments, progenitor cells are induced to differentiate progressively into various stages as described earlier with reference to FIG. 1. A tertiary medium may be prepared with differentiating factors such as a higher level of calcium, serum and/or TGF-(3. The medium may also include dexamethasone and cyclic adenosine monophosphate (cAMP) elevating agents, and other factors known to promote and sustain the growth of differentiating cells. Cell differentiation may also be promoted by addition of extracellular matrix, hydrogel or hydrocolloid substances or polymers that can assist the formation of cellular complexes. Such cells can be applied in various therapies.

Throughout the disclosure, where compositions are described as having, including, or comprising specific components, or where processes are described as having, including, or comprising specific process steps, it is contemplated that embodiments of the compositions may consist essentially of, or consist of, the recited components, and that embodiments of the processes also may consist essentially of, or consist of, the recited processing steps.

It should be understood that the order of steps or order for performing certain actions is immaterial so long as the invention remains operable. Moreover, two or more steps or actions may be conducted simultaneously.

The following examples are provided to illustrate embodiments and should not be interpreted in any way as limiting the scope of the claims. Those skilled in the art will recognize that various modifications can be made without departing from the spirit and scope of the disclosure.

### EXAMPLE 1

### Culture Conditions

Progenitor cells derived from human tissue are established by enzymatically dissociating the tissue of interest or mincing to form 1-2 mm2 tissue explants. If enzymatic digestion is used, enzymes such as collagenase, hyaluronidase, dispase, pronase, trypsin, elastase and chymotrypsin are preferred. Numerous methods of preparing a primary cell culture are known in the art.

Cultures are initiated by flattening and spreading a heterogeneous cell population onto a tissue culture substrate, such as a plate coated with Type I collagen. Typically, the majority of cells exhibit a large, spread epitheliod to fibroblastic appearance. The cells are then cultured in a chemically-defined culture medium that contains little or no calcium and very little or no growth factors. By chemically-defined conditions it is meant that the culture medium contains essentially no serum or organ extracts. If calcium is present in the culture medium, the concentration of calcium is preferably less than about 1 mM, e.g., between about 0.001 to about 0.9 mM. In another example, the calcium concentration is about 0.08 mM. If growth factor is present, its concentration is less than about 10 ng/mL, and preferably less than about 5 ng/mL, e.g., at about 1 ng/mL.

Single parenchymal progenitor cells and colonies of parenchymal progenitor cells are identified within the first 10 days. Usually, the colonies are visually distinct from other cells. Unlike most cells, the parenchymal progenitor cells remain small, rounded or hexagonal in shape. The progenitor cells are typically less than about 15 microns and have a dense appearance. Those cells are refractory and are readily-identified using phase-contrast microscopy. Moreover, the parenchymal progenitor cells can be identified by their active mitosis. Typically, colonies of parenchymal progenitor cells increase in number to become the predominant population in the primary culture within about 14 days. The cells are harvested by trypsinization when the loosely formed colonies and small dividing cells occupy about 50-70% of the cell culture surface. In one embodiment, the progenitor cells occupy about 80% of the cell culture surface.

The resulting progenitor population in a secondary culture is characterized as having a small size, a plating efficiency of about 40% or greater upon passage, and rapid cell division of about 36 hours or less. The progenitor cells are passaged for at least about 5 passages and can extend to about 13 passages, or more, depending on the split ratios used during passage. The cells typically achieve about 10 population doublings or greater. Cells maintain characteristics of tissue-specific progenitor cells, such as expression of lineage specific genes and genes developmentally associated with progenitor cells.

The progenitor cells have the ability to exhibit organotypic differentiation upon changing the culture conditions to an environment, i.e., a tertiary culture medium, that may contain factors that promote and/or support development and growth of differentiating cells. Examples of such factors include hydrocortisone, TGF-13, hepatocyte growth factor, or other factors that have been identified as effective in regulating embryonic organogenesis. Examples of other environmental conditions that can be introduced to the tertiary culture include the addition of an extracellular matrix to promote cluster formation or three-dimensional culture, the addition of calcium at a concentration greater than about 1.0 mM, or any method which allows cell-cell adhesion to occur and tissue architecture to develop. Any of these factors and conditions may be used together or in sequence to advance organogenesis depending on the tissue type.

### EXAMPLE 2

### Selection of Pancreatic Islet Cells

The selection of a population of pancreatic Islet progenitor cells is described below. However, that example is not intended to be limiting and progenitor cells can be derived from any organ or tissue containing parenchymal cells capable of regeneration such as the liver, gut, heart, cornea, skin, retina, inner ear, skeletal muscle, brain, or glands.

The endocrine progenitor cells are derived from either whole neonatal pancreas or isolated adult pancreatic islets. The cells are then cultured under stringent conditions to impose a stress condition on the cell culture in order to select for growth of an endocrine progenitor cell population. Once established, this population is propagated for multiple passages undifferentiated and thereby expanded for further experiments.

The stress-inducing culture medium of the invention allows for the establishment of primary cultures and facilitates the identification of a subpopulation of cells from these primary cultures that can then be serially passaged, thus providing for an expanded number of cells that could have therapeutic value. Preferably, the stress-inducing culture medium consists of a chemically defined medium without serum or growth factors. Cells grown from the pancreatic or islet tissue using this medium and culture methodology show a predominantly epithelial-like morphology and express cytokeratin markers characteristic of epithelial cells.

As the cells are expanded in culture, they are characterized by expression of markers associated with pancreatic progenitor cells, such as PDX 1. The homeodomain protein PDX1 is required at an early stage in pancreas development (Nature Genetics 15:106-110 (1997); Development 122:1409-1416 (1996)). As differentiated endocrine cells appear, they separate from the epithelium and migrate into the adjacent mesenchyme where they cluster.

PDX1 is later required for maintaining the hormone-producing phenotype of the B-cell by positively regulating insulin and islet amyloid polypeptide expression and repressing glucagon expression (Genes Dev 12:1763-1768 (1998)). PDX-1 is also required to regulate GLUT2 expression in 13-cells suggesting an important role in maintaining normal B-cell homeostasis.

Neurogenin-3, a member of the mammalian neurogenin gene family, has been established as a proendocrine gene (See, Proc. Natl. Acad. Sci. USA 97: 1607-11 (2000); Curr. Opin. Genet. Dev. 9:295-300 (1999)) and is considered a marker of islet progenitor cells during development (Development 129: 2447-57 (2002)). The progenitor cell characteristic of the islet-derived cell population expresses neurogenin-3.

The endocrine progenitor cells may be induced to differentiate using chemical or physical means, such as by supplementing the culture medium with an agent that promotes differentiation to insulin-producing beta cells or by inducing morphological changes such as cell cluster formation in the presence of extracellular matrix. The cells may also be induced to differentiate as a result of implantation into a permissive environment. For example, in vivo differentiation may be seen upon implantation under the kidney capsule, subcutaneously, or in the submucosal space of the small intestine.

### EXAMPLE 3

### Ingredients in Culture Media

A stringent, stress-inducing culture medium used for the primary culture contains no or essentially no serum or organ extracts.

A primary culture medium of the invention is provided with a nutrient base, which may or may not be further supplemented with other components. The nutrient base may include inorganic salts, glucose, amino acids and vitamins, and other basic media components. Examples include Dulbecco's Modified Eagle's Medium (DMEM); Minimal Essential Medium (MEM); M199; RPMI 1640; Iscove's Modified Dulbecco's Medium (EDMEM); Ham's F12, Ham's F-10, NCTC 109 and NCTC 135. A preferred base medium of the invention includes a nutrient base of either calcium-free or low calcium DMEM without glucose, magnesium or sodium pyruvate and with L-glutamine at 4.0 mM, and Ham's F-12 with 5 mM glucose in a 3-to-i ratio. The final glucose concentration of the base is adjusted to preferably about 5 mM. The base medium is supplemented with one or more of the following components known to the skilled artisan in animal cell culture: insulin or an insulin-like growth factor; transferrin or ferrous ion; triiodothyronine or thyroxin; ethanolamine and/or o-phosphoryl-ethanolamine, strontium chloride, sodium pyruvate, selenium, non-essential amino acids, a protease inhibitor (e.g., aprotinin or soybean trypsin inhibitor (SBTI)) and glucose.

In one example, no growth factor is added to the medium. In another example, the base medium is further supplemented with components such as non-essential amino acids, growth factors and hormones. For example, TGF-(3 is added as an apoptogen for promoting apoptosis of differentiated liver cells or TNFa is added as an apoptogen of differentiated islet, liver and epidermal cells. Defined culture media which can be useful in the present invention are described in U.S. Pat. No. 5,712,163 to Parenteau the disclosure of which of which is incorporated herein by reference. Titration experiments can be used to determine the appropriate concentrations for the supplements, as known by one skilled in the art. Examples of preferred concentrations are provided as follows:

A preferred concentration of insulin in the secondary medium is 5.0 μg/mL. Proinsulin, insulin-like growth factors such as IGF-1 or II may be substituted for insulin. Insulin-like growth factor as used herein means compositions which are structurally similar to insulin and stimulate the insulin-like growth factor receptors.

Preferably, ferrous ion is supplied by transferrin in the secondary medium at a concentration of from about 0.05 to about 50 μg/mL, a preferred concentration being about 5 μg/mL.

Triiodothyronine is added to maintain rates of cell metabolism. It is preferably present at a concentration of from about 2 to about 200 pM, more preferably at about 20 pM.

Either or both ethanolamine and o-phosphoryl-ethanolamine may be used in the practice of the present invention. Both are phospholipids that function as precursors in the inositol pathway and in fatty acid metabolism. Supplementation of lipids that are normally found in serum may be necessary in a serum-free medium. Either or both ethanolamine and o-phosphoryl-ethanolamine are provided to the media at a concentration range of preferably between about 10-6 M to about 10-2 M, more preferably between about 10′ M.

Selenium may be used at a concentration between about 10-9 M to about 10-7 M, preferably at about 5×10-8 M. And amino acid L-glutamine or its substitute may be used at a concentration between about 1 mM to about 10 mM, preferably at about 6 mM.

When preparing the secondary medium for serial passage of progenitor cells, other components may be added to the media, depending upon, e.g., the particular cell being cultured, including but not limited to, epidermal growth factor (EGF), transforming growth factor alpha (TNF-a), keratinocyte growth factor (KGF), and basic fibroblast growth factor (bFGF). EGF as an optional component in a secondary medium may be used at a concentration as low as 1 ng/mL.

A preferred embodiment of the secondary medium includes: a base 3:1 mixture of DMEM (no glucose, no calcium and 4 mM L-glutamine) and Ham's F-12 medium supplemented with the following components to achieve the final concentration indicated for each component: 6 mM L-glutamine (or equivalent), 1 ng/mL EGF, 1×10-4 M ethanolamine, 1×10-4 M o-phosphorylethanolamine, 5 μg/mL insulin, 5 μg/mL transferrin, 20 pm triiodothyronine, 6.78 ng/mL selenium, 24.4 μg/mL adenine, 1 mM strontium chloride, 100 mM sodium pyruvate, 10 mM non-essential amino acids, and 5 mM to 20 mM glucose depending on cell type, eg. islet, liver or epidermal.

While the cell population propagated according to the invention comprises a pool of progenitor cells at one stage, further commitment to differentiation and organ development may be induced. A tertiary medium allows the progenitor cells to generate a majority of transit amplifying cells and advance organogenesis when desired. A preferred embodiment of the medium for the generation of transit amplifying cells includes a base mixture of 1:1 DMEM (no glucose, no calcium and 4 mM L-glutamine) and Ham's F-12 medium supplemented with the following components to achieve the final concentration indicated for each component: 6 mM L-glutamine (or equivalent), 10 ng/mL EGF or HGF or both (depending on cell type), 1×10-4 M ethanolamine, 1×10-4 M o-phosphorylethanolamine, 5 gg/mL insulin, 5 μg/mL transferrin, 20 pm triiodothyronine, 6.78 ng/mL selenium, 24.4 gg/mL adenine, 100 mM sodium pyruvate, 2×10-9 progesterone, 1.1 μM hydrocortisone, 0.08 mM calcium chloride and 9 ng/mL forskolin and a glucose concentration between 5-20 mM depending on the cell type. In can be beneficial to adjust the concentration higher (e.g. islet) or lower (e.g., epidermis) relative to theoptimal secondary medium during this phase to aid organogenesis.

Progenitor cells may be plated at a moderate density of between 1,000 to 5,000 cells per cm2 in this medium on a collagen-coated plastic surface and cultured for at least one passage. The cell population may be used as is or further differentiation may be initiated.

Where further differentiation is desired, the cells are transferred to conditions where forskolin is removed from the tertiary medium and the calcium concentration is increased, e.g., to about 1.88 mM. Other changes to the culture environment also may be included at this time, e.g., addition of an extracellular matrix component. Some of the changes in the environmental condition depend on the tissue type, e.g. epidermal cells may be cultured at an air-liquid interface, islet cells may be cultured in a matrix condition that promotes cluster formation, and hepatocyte cells may be cultured in a 3-dimensional substrate that promotes cord formation.

A typical way of preparing media useful for the present invention is set forth below. However, components of the present invention may be prepared using other conventional methodology with or without substitution in certain components with an analogue or functional equivalent. Also, concentrations for the supplements may be optimized for cells derived from different species and cell lines from different organisms due to factors such as age, size and health. Titration experiments can be performed with varying concentrations of a component to arrive at the optimal concentration for that component.

The medium, whether primary, secondary or tertiary, is prepared under sterile conditions, starting with base medium and components that are bought or rendered sterile through conventional procedures, such as filtration. Proper aseptic procedures are used throughout the Examples. DMEM and F-12 are combined and the individual components are then added to complete the medium. Stock solutions of all components can be stored at 20 oC, with the exception of the nutrient source that can be stored at 4 oC.

A vessel suitable for animal cell or tissue culture, e.g., a culture dish, flask, or roller bottle, is used to culture the endocrine progenitor cells. Materials such as glass, stainless steel, polymers, silicon substrates, including fused silica or polysilicon, and other biologically compatible materials may be used as cell growth surfaces. The cells of the invention may be grown on a solid surface or a porous surface, such as a porous membrane, that would allow bilateral contact of the medium to the cultured cells. In addition, the cell growth surface material may be chemically treated or modified, electrostatically charged, or coated with biological agents such as with peptides or matrix components. The preferred growth surface for carrying out the invention is a conventional tissue culture surface coated with Type I collagen.

The cultures are preferably maintained between about 34 ° C. to about 38 ° C., more preferably 37° C., with an atmosphere between about 5-10% CO2 and a relative humidity between about 80 to 90%. An incubator is used to sustain environmental conditions of controlled temperature, humidity, and gas mixture for the culture of cells.

Medium used during the first step in progenitor cell activation from a resident progenitor cell can be harvested and used to promote activation of progenitor cells still residing in the expanded culture. Similarly, conditioned medium from proliferating later passage cells can be used to support proliferation of progenitor cells plated at low density. The conditioned medium can comprise from 10-50% of the nutrient medium. Alternatively, a more specialized conditioned supplement is created by removing the common heparin-binding growth factors, concentrating, and desalting the harvested medium. The concentrated supplement can be used at a concentration equivalent to the original starting material.

### EXAMPLE 4

### Generation of ADAP samples for Genomic and Proteomic Analysis

Population of ADAP cells can be obtained from different tissues using the procedures described herein. ADAP cells from one organ can then be compared to differentiated cells in the adult parenchyma and ADAP derived from different organs.

In one example, pancreatic islets are isolated using established methods. Isolation of human islets is performed using the semi-automated method originally proposed by Ricordi (Diabetes 37:413-420, 1988). Procured organs are distended by intraductal infusion of Liberase HI (Roche Molecular Biosciences, Indianapolis Ind.) or Serva collagenase (Crescent Chemical, Brooklyn, N.Y.). After a process of continuous digestion for approximately 12 to 30 min, the tissue is collected into about 8 liters of Hanks solution and washed. Free islets are separated from the other tissue using a continuous gradient of EuroFicoll in a Cobe 2991 cell separator (Cell Tiss Res. 310:51-58, 2002).

Islets are washed in Hank's buffered saline and resuspended in a nutritional medium containing:
DMEM (calcium-free, 1 g/L glucose) and Ham's F 12 mixed 3:1 10
Soybean trypsin inhibitor (12.5 pg/mL) or aprotinin (25 gg/mL)
Gentamicin 50 μg/mL

Islets are then plated at a density of about 3,000 Islet Equivalents per 100 mm cell culture dish which has been treated with a Type I collagen solution at a concentration of 5 p.g/cm2 for 30 minutes. A portion of the Islets from each isolation is aliquoted into approximately 2-3×106 cells/aliquot (estimating 800- 1000 cells/IE) and reserved for comparative analysis against ADAP derived from the same donor and different donors. Medium is changed every 3-4 days until the more differentiated cells begin to release from the dish and the progenitor colonies appear and proliferate to become the predominant cell type. Plates are washed in buffered saline to remove any remaining islet remnants, if present. Pre-confluent progenitor cells are either extracted on the plate for genomic analysis or harvested for proteome analysis using a trypsin substitute to retain membrane proteins (Hyclone or GIBCO). The suspension is centrifuged, washed in buffered saline, recentrifuged and resuspended in an appropriate buffer for further processing. Cell culture supernatant is retained at days 4, 7, 10 and 14 (or the last day before passage) and saved for proteomics analysis.

In a second preparation, a skin sample is obtained from clean excisions. Fat and deep dermis is removed by dissection and the skin is minced into 2 mm2 explants. Some explants are reserved for overnight incubation in 0.2% trypsin to dissociate the epidermis from the dermis. Following incubation, the epidermis is removed with forceps, washed in PBS and saved for comparative genomic and proteomics analysis. The remaining explants are plated onto 100 mm2 tissue culture plates that have been coated for 30 minutes with a 5 μg/cm2 of a Type I collagen solution. Ten to 14 explants are placed dermis side down in each dish and air-dried until tacky before medium is added to cover the explants. The medium contains: DMEM (calcium-free, 4.5 g/L glucose) and Ham's F12 mixed 3:1

| | | |
|---|---|---|
| 15 Transferrin | 5 | μg/mL |
| Tri-iodothyronine | 20 | pM |
| Insulin | 5 | p · g/mL |
| Selenium | 3 × 10″8 | M |
| Ethanolamine | 1 × 104 | M |
| 20 Phosphoethanolamine | 1 × 104 | M |
| L-Glutamine | 1.5 | mM |
| Glutamax | 4.5 | mM |
| Strontium chloride | 1.0 | mM |
| Adenine | 0.18 | mM |
| 25 Non-essential amino acids | 100 | μM |
| Soybean trypsin inhibitor (12.5 μg/mL) or | 50 | μg/mL |
| aprotinin (25 μg/mL) Gentamicin | | |

The cultures are fed every 3-4 days with the defined nutrient medium. Cell culture 30 supernatant is retained at days 4, 7, 10 and 14 (or the last day before passage) and saved for proteomics analysis. Cultures are harvested when the proliferative epithelial outgrowths exhibit a near uniform morphology of small rapidly proliferating cells and are at least 60% confluent, between approximately 12 and 14 days. Remnants of original explants, if present, are removed by aspiration prior to harvesting of ADAP cells. Cells on plates are either extracted on the plate for genomic analysis or are harvested for proteomics analysis using a trypsin substitute (Hyclone or GIBCO) to better retain surface membrane proteins. Cells are centrifuged, resuspended in Hanks Balanced Salt Solution and counted using a hemocytometer. Cells are then aliquoted and resuspended in the appropriate buffer for further processing.

### EXAMPLE 5

Comparative Genomic Analysis Using Suppression Subtractive Hybridization (SSH) of ADAP and Primary Tissues

Progenitor cells in their active state should be rare to non-existent in the mature tissue providing an opportunity to use SSH to discover genes novel to the ADAP phenotype. This is accomplished by delineating differences in gene expression between mature tissue and ADAP and similarities among ADAP cells derived from different organs.

Adherent islet- and skin-derived progenitor cells are lysed and homogenized in Trizol reagent (Invitrogen Life Technologies). Isolated mature islets and epidermis reserved in Example 4 are also lysed and homogenized as samples. Total RNA is extracted following manufacturer's instructions. Poly(A+) MRNA is isolated from the total RNA of each sample using Oligotex MRNA spin columns (Qiagen) according to manufacturer's instructions. SSH is a PCR-based subtraction method used to target differentially expressed cDNA fragments while simultaneously suppressing nontarget DNA amplification. SSH is performed as described by Diatchenko et al., Proc. Natl. Acad. Sci. USA 93:6025 (1996), using the following combinations of drivers and targets to find cDNA specific to the ADAP phenotype. Differentially expressed cDNAs between mature Islets and Islet ADAP are determined by using mature Islet cDNA as the driver and Islet ADAP as the target. Differentially expressed cDNAs between mature epidermis and epidermal ADAP are determined by using mature epidermal cDNA as the driver and epidermal ADAP as the target. The samples are hybridized twice and then undergo PCR amplification. The cDNA common to the driver and the target is suppressed in the amplification. The subtracted cDNA from the islet comparison and the skin comparison are labeled and then hybridized to Northern blots of the other ADAP type to determine the degree of similarity between ADAP populations. Cloning of the subtracted ADAP cDNA are used to generate progenitor-specific cDNA libraries. ADAP cells derived from multiple donors of the same tissue type and of different tissue types can be compared using Northern blots and further subtractive hybridizations can be performed between ADAP cells under conditions that retain the representation of quantitatively different cDNA in the final SSH products by omitting a second hybridization step allowing quantitatively regulated cDNA to amplify in subsequent PCR. SSH in combination with the ability to sample ADAP from multiple tissues provides a way to distinguish key regulatory genes associated with progenitor cell phenotype and behavior.

### EXAMPLE 6

### Comparative Analysis of Progenitor Signaling Using Isotope Coded Affinity Tag (ICAT)

The cells are harvested and split into 2-3×106 cells per aliquot and centrifuged. Each aliquot is resuspended in 80 μL of 50 mM Tris Buffer, pH 8.0, containing 0.1% SDS and 0.02% Triton. The samples are lysed in a sonic water bath for 30 minutes followed by rapidly freezing and thawing 3 times. Cellular debris is removed by centrifugation @17,000×g for 1 hour. Total protein of each lysate is determined using a BCA assay and the protein concentration is adjusted to 1.5 mg/mL using the Tris buffer containing 0.1% SDS.

One hundred micrograms of total protein from epidermal and islet ADAP cells are individually reduced using tris(2-carboxyethyl)phoshine hydrochloride, then alkylated with ICAT reagent (Applied Biosystems) which labels cysteine-containing peptides. The light form of ICAT is used for the epidermal ADAP and the heavy form of ICAT is used for the islet ADAP. The differing mass of ICAT forms enables common proteins to be quantitatively distinguished as light and heavy doublet peaks. Samples are mixed and separated using 1D SDS-PAGE, stained to visualize the proteins. Comparison with lanes of unmixed ADAP proteins and 2D gels are used to identify MW ranges of greatest homology. Lanes are cut into segments and selected segments subjected to in-gel tryptic digestion. A full mass spectrum of the tryptic digest is used to identify peptide doublets differing by four or eight mass units indicative of isotope binding. Selected peptides are then fractionated using μLC/MS/MS for sequence determination.

### EXAMPLE 7

Discovery and Partial Purification of Regenerative Signaling Molecules Using ADAP Cells

The method of the invention relies on autocrine and paracrine responses of the cells and tissues for derivation of the ADAP population. Molecules involved in the signaling emitted from the primary explants and the activated, proliferating ADAP cells should be present in the conditioned nutrient medium. Novel, non-heparin binding molecules that stimulate growth of ADAP cells are of particular interest in this experiment.

The media samples from the final collection during the in vitro derivation and growth of the ADAP cells from a single tissue type are pooled and concentrated by filtration using 3000NMWL regenerated cellulose membrane (Millipore) in a stirred cell concentrator at 4° C. Concentrated media are mixed 1:1 with 50 mM sodium phosphate containing 0.15 M sodium chloride, pH 8.0 and loaded onto a heparin sepharose-CL6B column to bind more common heparin binding growth factors. The passthrough containing the non-heparinbinding proteins and peptides are collected and analyzed using 2D-gel electrophoresis. Media components such as phenol red are removed using preparative reversed-phase HPLC. Peak fractions are detected by UV absorbance and pooled for analysis. Differential analysis between proteins derived from two different ADAP populations (epidermal and islet for example) are used to distinguish signaling molecules most likely to be involved in mechanisms of renewal.

Protein concentration is determined using a PlusOne 2-D Quant Kit (Amersham). Samples are concentrated if necessary. Fifty jig of protein from ADAP-1 and ADAP-2 are labeled with Amersham Biosciences Cy-3 and Cy-5 N-hydroxysuccinimidyl ester dyes. An internal standard is formed by using pooled samples of 25 μg of each sample labeled with Cy-2. All samples are mixed together and diluted with an equal volume of sample buffer. Isoelectric focusing is performed using Immobiline (IPG) Drystrips pH 3-10 (Amersham). SDS PAGE is performed using, e.g., a 12.5% polyacrylamide gel. Following electrophoresis, the gel is scanned using an Amersham Typhoon 9400 imager. Image analysis is performed using the differential in-gel analysis (Amersham DeCyder software) to determine spots that are statistically different. Replicate samples are processed to confirm differences and similarities between samples. The Cy-2 marker permits normalization between gels. The gel is fixed and stained with Sypro Ruby to facilitate excision of spots of interest. The protein profiles are also compared to proteins derived from human fibroblast conditioned medium to further eliminate spots unlikely to be related to ADAP signaling.

The disclosed embodiments may be embodied in other specific forms without departing from the spirit or essential characteristics thereof. The foregoing embodiments are therefore to be considered in all respects illustrative rather than limiting on the claims. All changes which come within the meaning and range of equivalency of the claims are intended to be embraced therein.

What is claimed is:

1. A method of identifying a biological process associated with non-fetal progenitor cells, the method comprising the steps of:
providing at least one substantially pure population of non-fetal progenitor cells, wherein the cell population originates from a differentiated tissue; and
identifying a biological process present in the population of non-fetal progenitor cells but absent or substantially absent in a differentiated cell or a substantially pure population of non progenitor cells.

2. A method of identifying a biological process associated with non-fetal progenitor cells, the method comprising the steps of:
providing at least one substantially pure population of non-fetal progenitor cells, wherein the cell population originates from a differentiated tissue;
administering a test molecule to the population of non-fetal progenitor cells; and
identifying a biological process affected in the population of non-fetal progenitor cells upon administration of the test molecule.

3. The method of claim 2 further comprising identifying a biological process affected in the population of non-fetal progenitor cells but not affected in a differentiated cell or a substantially pure population of non progenitor cells.

4. The method of claim 1, wherein the non progenitor cells are fibroblasts.

5. The method of claim 1, wherein the progenitor cell population originates from parenchymal tissue.

6. The method of claim 1, wherein the progenitor cell population originates from pancreatic islets, liver hepatocytes, or epidermal cells.

7. The method of claim 2, wherein the biological process affected is the level of expression of a gene, or the amount of a gene product in the non-fetal progenitor cells upon administration of the test molecule.

8. The method of claim 7, wherein the gene product is a messenger RNA or a protein.

9. The method of claim 1, further comprising comparing levels of protein and gene expression in progenitor cells to those in more differentiated cells of the same origin.

10. The method of claim 1, further comprising providing a second substantially pure population of non-fetal progenitor cells originating from a differentiated tissue different from that of the first population of progenitor cells, and identifying a biological process present in both population of non-fetal progenitor cells but absent or substantially absent in the population of non progenitor cells or differentiated cells.

11. The method of claim 2, further comprising:
providing a second substantially pure population of non-fetal progenitor cells originating from a differentiated tissue different from that of the first population of progenitor cells;
administering the test molecule to the second population of progenitor cells; and (c) identifying a biological process affected in both populations of progenitor cells upon administration of the test molecule.

12. The method of claim 11, further comprising identifying at least one test molecule and related biological process common to propagation or differentiation of progenitor cells of more than one origin.

13. The method of claim 11, further comprising comprises identifying at least one test molecule and related biological process specific for propagation or differentiation of progenitor cells of one origin.

14. The method of claim 1 further comprising the step of creating a database containing data indicative of the biological process present or affected in the non-fetal progenitor cells.

15. A database produced by the method of claim 14.

16. A method for assaying the activity of a test molecule on non-fetal progenitor cells, the method comprising the steps of:
providing a substantially pure population of non-fetal progenitor cells in a defined cell culture medium;
administering a test molecule to the population of non-fetal progenitor cells; and
determining if the test molecule modulates a biological process within cells of the cell population.

17. The method of claim 16, wherein the population of progenitor cells originates from parenchymal tissue.

18. The method of claim 16, wherein the test molecule is assayed on a plurality of non-fetal progenitor cell populations for any modulatory effect, each cell population originating from a different differentiated tissue.

19. The method of claim 16, wherein at least one of the progenitor cell populations originates from pancreatic islets, liver hepatocytes, or epidermal cells.

20. A cDNA library comprising a population of DNA molecules representative of genes expressed in a progenitor cell originated from a mature tissue.

21. The cDNA library of claim 20, wherein the tissue is liver tissue, pancreas tissue, or epidermis.

22. A cDNA library comprising a population of DNA molecules representative of genes commonly expressed in at least two populations of progenitor cells, wherein each population has -originated from a different mature tissue.

23. The cDNA library of claim 22, wherein at least one population of the progenitor cells has originated from the liver, the pancreas, or the epidermis.

24. The method of claim 3, wherein the non progenitor cells are fibroblasts.

25. The method of claim 2, wherein the progenitor cell population originates from parenchymal tissue.

26. The method of claim 2, wherein the progenitor cell population originates from pancreatic islets, liver hepatocytes, or epidermal cells.

27. The method of claim 2, further comprising comparing levels of protein and gene expression in progenitor cells to those in more differentiated cells of the same origin.

28. The method of claim 2 further comprising the step of creating a database containing data indicative of the biological process present or affected in the non-fetal progenitor cells.

29. A database produced by the method of claim 28.
